(19) United States
(12) Patent Application Publication
(10) Pub. No.: US 2024/0298943 A1
(43) Pub. Date: Sep. 12, 2024

(54) BIOSIGNAL SENSING DEVICE

(71) Applicant: LOOXID LABS INC., Daejeon (KR)

(72) Inventors: Hong Gu LEE, Seoul (KR); Jung Ho PARK, Daejeon (KR); Yong Wook CHAE, Seoul (KR)

(73) Assignee: LOOXID LABS INC., Daejeon (KR)

(21) Appl. No.: 17/768,854

(22) PCT Filed: Nov. 13, 2020
(86) PCT No.: PCT/KR2020/016028

§ 371 Date: Apr. 14, 2022

(30) Foreign Application Priority Data
Nov. 15, 2019 (KR) 10-2019-0146602

(51) Int. Cl. A61B 5/16; A61B 5/00; G06F 1/16

(52) U.S. Cl. CPC A61B 5/165; A61B 5/6803; G06F 1/16; A61B 2562/04; A61B 2562/164; A61B 2562/166

(57) ABSTRACT

Disclosed is a biosignal sensing device capable of measuring at least one biosignal while being in contact with a user's skin. The biosignal sensing device includes a sensor configured to detect at least one biosignal while being in contact with a user's skin, and the sensor is deformed to a shape corresponding to a contact portion and comes into close contact with the contact portion.

### TECHNICAL FIELD

The present invention relates to a biosignal sensing device. More specifically, it relates to a biosignal sensing device capable of detecting at least one biosignal while being in contact with a user's skin.

### BACKGROUND ART

In general, a head-mounted display (HMD) device refers to a display device having a structure that may be worn on a user's head. The head-mounted display is configured to provide the user with virtual reality (VR) images to enable the user to have a spatial, temporal experience similar to an actual experience.

The head-mounted display device includes a main body provided in the form of a goggle so as to be worn on the user's eye area, and a wearing part connected to the main body and provided in the form of a band in order to fix the main body to the user's head. In this case, the main body has, as a means for outputting a virtual reality image. A portable terminal device such as a smartphone is installed in the main body, or a display device such as a monitor connected to a PC is installed.

However, the head-mounted display device is manufactured to allow the user to only visually check the image outputted from an image output means installed on the portable terminal device. For this reason, there are problems in that utilization deteriorates, and types of contents provided to the image output means are highly restricted.
Related Patent Document: (Patent Document 1) Korean Patent Application Laid-Open No. 10-2017-0094883

### SUMMARY OF DISCLOSURE

The present invention has been made to solve the above-mentioned problems. An object of the present invention is to provide a biosignal sensing device capable of measuring and analyzing a biosignal of a user who uses contents outputted from the head-mounted display device, and the biosignal sensing device being capable of determining the user's health state and emotional/perceptional state.

Technical problems of the present invention are not limited to the aforementioned technical problems, and other technical problems, which are not mentioned above, may be clearly understood by those skilled in the art from the following descriptions.

To achieve the above-mentioned object, a biosignal sensing device according to an embodiment of the present invention includes a sensor configured to detect at least one biosignal while being in contact with a user's skin, and the sensor is deformed to a shape corresponding to a contact portion and comes into close contact with the contact portion.

The sensor may be disposed on the user's forehead portion and measure the at least one biosignal.

The sensor may include: a plurality of electrodes configured to detect the at least one biosignal while being in contact with the user's skin; a flexible printed circuit board electrically connected to the plurality of electrodes and configured to be deformed to correspond to a shape of the contact portion; and a connection terminal electrically connected to the flexible printed circuit board and an external device.

The plurality of electrodes may include: a reference electrode; a plurality of active electrodes disposed to be opposite to one another and provided at one side and the other side of the reference electrode based on a horizontal direction with respect to the reference electrode; and a plurality of ground electrodes disposed to be opposite to one another and provided at one side and the other side of the reference electrode based on the horizontal direction with respect to the reference electrode, and the plurality of active electrodes may be disposed at least two positions based on vertical direction.

The plurality of active electrodes may include: a first active electrode disposed above the reference electrode based on the vertical direction; a second active electrode disposed below the reference electrode based on the vertical direction; and a third active electrode disposed below the second active electrode based on the vertical direction, and the first active electrode, the second active electrode, and the third active electrode may be disposed at different positions based on the horizontal direction.

The biosignal sensing device may further include: a bracket configured to be coupled to a head-mounted display device; and a support pad coupled to the bracket and configured to bring the sensor, coupled to one side into close contact with the user's skin.

The bracket may include: a support plate coupled to the support pad and having a curved shape; and a coupling member extending from the support plate and configured to be coupled to the head-mounted display device.

The support pad may have a concave-convex structure configured to elastically support the plurality of electrodes.

The support pad may include: a base coupled to the bracket and configured to maintain a shape corresponding to the bracket; and a plurality of pressing protrusions provided on the base so that the plurality of electrodes each disposed on one surface of each of the plurality of pressing protrusions is disposed at positions spaced apart from one surface of the base, the plurality of pressing protrusions being configured to elastically support the plurality of electrodes and bring the plurality of electrodes into close contact with the user's skin.

The support pad may be made of a material having elasticity, and the plurality of pressing protrusions may each have a shell structure having a vacant internal space.

The biosignal sensing device may further include a control unit electrically connected to the sensor and configured to receive the at least one biosignal detected by the sensor, process and convert the received biosignal, and transmit the biosignal to the external device.

According to the embodiment of the present invention, the biosignal sensing device may be applied to the head-mounted display device to measure and analyze a user's biosignal using contents outputted from the head-mounted display device. The biosignal sensing device may determine the user's health state and emotional/perceptional state through this.

In addition, according to the embodiment of the present invention, the biosignal sensing device may be applied to the various head shapes and, acquire the biosignals in the entire region of the prefrontal lobe. The biosignal sensing device may accurately measure the biosignal while being in completely close contact with the skin.

In addition, according to the embodiment of the present invention, the support pad made of silicone is used to press the sensor. The support pad is deformed to a shape corresponding to the contact portion, such that the sensor may be in completely close contact with the contact portion.

In addition, the support pad has the concave-convex structure having elasticity and presses the sensor. Therefore, even though a shape or size of the head of the user changes, the support pad may bring the sensor into completely close contact with the skin of the user, thereby maintaining the state in which the electrode and the skin are in contact with each other. Therefore, the quality of the detected biosignal may be maintained in the highest state.

In addition, the concave-convex structure configured to elastically support the electrode is configured as a shell structure, it possible to minimize the force applied to press the user's skin and minimize fatigue caused by the pressing.

The effects according to the present invention are not limited to the above-mentioned effects, and more various effects are included in the present invention.

### BRIEF DESCRIPTION OF THE DRAWINGS

FIG. 1 is a view schematically illustrating a state in which a user wears a head-mounted display device to which a biosignal sensing device according to an embodiment of the present invention is coupled.

FIG. 2 is a perspective view illustrating the head-mounted display device to which the biosignal sensing device according to the embodiment of the present invention is coupled.

FIG. 3 is an exploded perspective view illustrating the biosignal sensing device according to the embodiment of the present invention.

FIG. 4 is a front view illustrating a sensor of the biosignal sensing device according to the embodiment of the present invention.

FIG. 5 is a rear view illustrating the sensor of the biosignal sensing device according to the embodiment of the present invention.

FIG. 6 is a view schematically illustrating a change of a pressing protrusion when a support pad of the biosignal sensing device according to the embodiment of the present invention brings the sensor into close contact with a user's skin.

FIG. 7 is a perspective view illustrating a state of the support pad of the biosignal sensing device according to the embodiment of the present invention when viewed from the front side.

FIG. 8 is a perspective view illustrating a state of the support pad of the biosignal sensing device according to the embodiment of the present invention when viewed from the rear side.

FIG. 9 is a view schematically illustrating a state in which a user wears a head-mounted display device to which a biosignal sensing device according to another embodiment of the present invention is coupled.

FIG. 10 is a rear perspective view illustrating the head-mounted display device to which the biosignal sensing device according to another embodiment of the present invention is coupled.

FIG. 11 is an exploded perspective view illustrating the biosignal sensing device according to another embodiment of the present invention.

FIG. 12 is a front view illustrating a sensor of the biosignal sensing device according to another embodiment of the present invention.

FIG. 13 is a rear view illustrating the sensor of the biosignal sensing device according to another embodiment of the present invention.

FIG. 14 is a perspective view illustrating a state in which a coupling member of a first bracket of the biosignal sensing device according to another embodiment of the present invention is separated from a support plate when viewed from the front side.

FIG. 15 is a perspective view illustrating a state in which the coupling member of the first bracket of the biosignal sensing device according to another embodiment of the present invention is separated from the support plate when viewed from the rear side.

FIG. 16 is a perspective view illustrating a second bracket of a bracket of the biosignal sensing device according to another embodiment of the present invention.

FIG. 17 is a perspective view illustrating a state of the support pad of the biosignal sensing device according to another embodiment of the present invention when viewed from the front side.

FIG. 18 is a perspective view illustrating a state of the support pad of the biosignal sensing device according to another embodiment of the present invention when viewed from the rear side.

FIG. 19 is a perspective view illustrating a state of a buffer pad of the biosignal sensing device according to another embodiment of the present invention when viewed from the front side.

FIG. 20 is a view perspective view illustrating a state of the buffer pad of the biosignal sensing device according to another embodiment of the present invention when viewed from the rear side.

FIG. 21 is a view schematically illustrating changes of a pressing protrusion and a buffer protrusion when the support pad of the biosignal sensing device according to another embodiment of the present invention brings the sensor into close contact with the user's skin.

FIG. 22 is a perspective view a state of an ear clip of the biosignal sensing device according to another embodiment of the present invention when viewed from the front side.

FIG. 23 is a view illustrating a cross-section of a part of the ear clip of the biosignal sensing device according to another embodiment of the present invention.

FIG. 24 is a view illustrating a state of a control unit of the biosignal sensing device according to another embodiment of the present invention when viewed from the left side.

FIG. 25 is a view illustrating a state of the control unit of the biosignal sensing device according to another embodiment of the present invention when viewed from the right side.

### DETAILED DESCRIPTION OF THE EMBODIMENT

Advantages and features of the present invention and methods of achieving the advantages and features will be clear with reference to embodiments described in detail below together with the accompanying drawings. However, the present invention is not limited to the embodiments disclosed herein but will be implemented in various forms. The embodiments of the present invention are provided so that the present invention is completely disclosed, and a person with ordinary skill in the art to which the present invention pertains can fully understand the scope of the present invention. the present invention will be defined only by the scope of the appended claims.

Shapes, areas, ratios, angles, numbers, and the like illustrated in the drawings for explaining the embodiments of the present invention are just exemplarily illustrated, and the present invention is not limited to the contents illustrated in the drawings. Throughout the specification, the same reference numerals denote the same constituent elements. In addition, in the description of the present invention, the specific descriptions of publicly known related technologies will be omitted when it is determined that the specific descriptions may unnecessarily obscure the subject matter of the present invention. The terms “comprise,” “have,” or “include” used in the present invention may mean that other constituent elements can be added unless these terms are used with the term “only”. Unless otherwise particularly and clearly stated, the singular expressions used herein are intended to include the plural expressions.

Unless otherwise separately and explicitly stated, analyses of constituent elements are interpreted as including error ranges.

In the description of a positional relationship, for example, when a positional relationship between two components is described by using terms ‘on,’ ‘above,’ ‘below,’ ‘at a lateral side of,’ and the like, one or more components may be positioned between the two components when term ‘immediately,’ or ‘directly’ is not used.

In addition, terms “first”, “second”, and the like may be used to describe various components, but the components are of course not limited by these terms. These terms are merely used to distinguish one component from another component. Therefore, the first component mentioned hereinafter may be the second component within the technical spirit of the present invention.

Throughout the specification, the same reference numerals denote the same constituent elements.

The area and thickness of each component illustrated in the drawings are shown for ease of description, but the present invention is not necessarily limited to the area and thickness of the illustrated component.

Respective features of several embodiments of the present invention may be partially or entirely coupled to or combined with each other, and various technical cooperation and operations may be made, and the respective embodiments may be carried out independently of each other or carried out together correlatively.

Hereinafter, various embodiments of the present invention will be described in detail with reference to the accompanying drawings.

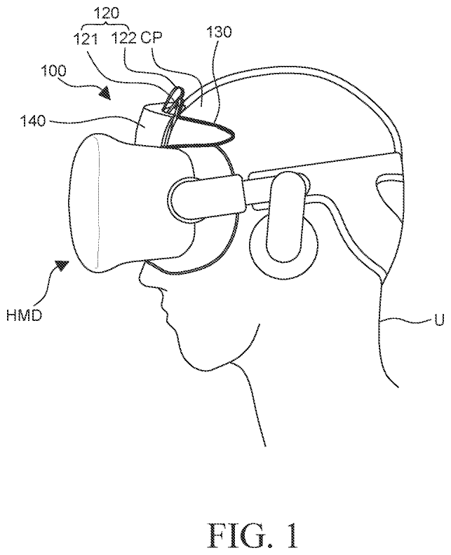

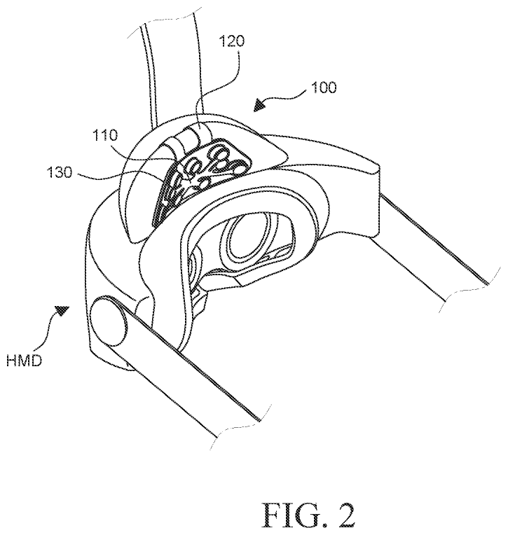

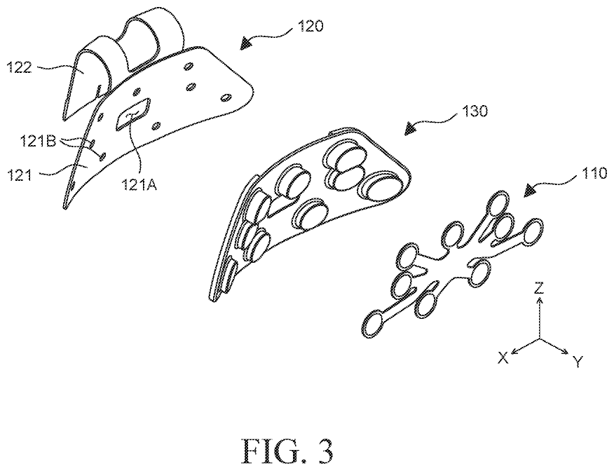

FIG. 1 is a view schematically illustrating a state in which a user wears a head-mounted display device to which a biosignal sensing device according to an embodiment of the present invention is coupled, FIG. 2 is a perspective view illustrating the head-mounted display device to which the biosignal sensing device according to the embodiment of the present invention is coupled, and FIG. 3 is an exploded perspective view illustrating the biosignal sensing device according to the embodiment of the present invention.

Referring to FIGS. 1 and 2, a biosignal sensing device 100 according to an embodiment of the present invention may be detachably mounted on a head-mounted display device HMD. And the biosignal sensing device 100 detect a biosignal while being in contact with skin of the user who wears the head-mounted display device HMD.

Specifically, the biosignal sensing device 100 coupled to the head-mounted display device HMD may be disposed at an upper side of the head-mounted display device HMD. When the user U wears the head-mounted display device HMD, the biosignal sensing device 10 may detect at least one biosignal while being in contact with a forehead portion of a user U.

The head-mounted display device HMD may have a structure that can be worn on the head of the user U. Further, the head-mounted display device HMD may provide the user U with virtual reality (VR) images to enable the user U to have a spatial, temporal experience similar to an actual experience. For example, the head-mounted display device HMD may include a wearing part and a display part. The wearing part is provided in the form of a band capable of being worn on a periphery of the head of the user U. The wearing part may have an inner diameter adjusting means provided at one side thereof, and the inner diameter adjusting means may be used to adjust an inner diameter thereof. The display part is coupled to the wearing part, and configured to be in close contact with the user's face. The display part may provide the user U with the virtual reality images.

In the present embodiment, the configuration has been described in which the biosignal sensing device 100 has the structure detachably mounted on the head-mounted display device HMD. However, the biosignal sensing device 100 according to the embodiment of the present invention may not be necessarily detachably mounted only on the head-mounted display device HMD. The biosignal sensing device 100 may formed in structure that can be used independently while not coupled to the head-mounted display device HMD.

Referring to FIGS. 1 to 3, the biosignal sensing device 100 includes a sensor 110.

The sensor 110 is supported by a support pad 130 coupled to a bracket 120. The sensor 110 detects at least one biosignal while being disposed on the forehead portion of the user U, who wears the head-mounted display device HMD, and being in contact with the skin of the user U.

Specifically, the sensor 110 may measure a brainwave signal of a prefrontal lobe while being in contact with the forehead portion of the user U. However, the sensor 110 may not necessarily measure only the brainwave signal of the prefrontal lobe while being in contact with the forehead portion of the user U. The sensor 110 may be configured to detect various biosignals while being in contact with various body portions of the user U.

In addition, when the sensor 110 is in contact with the body of the user U, the sensor 110 may be deformed to a shape corresponding to a contact portion CP. And the sensor 110 may be in completely adhered to the contact portion CP.

Specifically, the sensor 110 may be provided in the form of a film having flexibility so that the sensor 110 may be bent or curved and thus be deformed to a shape corresponding to the contact portion CP. Therefore, even though a shape of the contact portion CP is changed, the sensor 110 may be deformed to a shape corresponding to the contact portion CP, such that the sensor 110 may always maintain in a state being completely close contact with the contact portion CP.

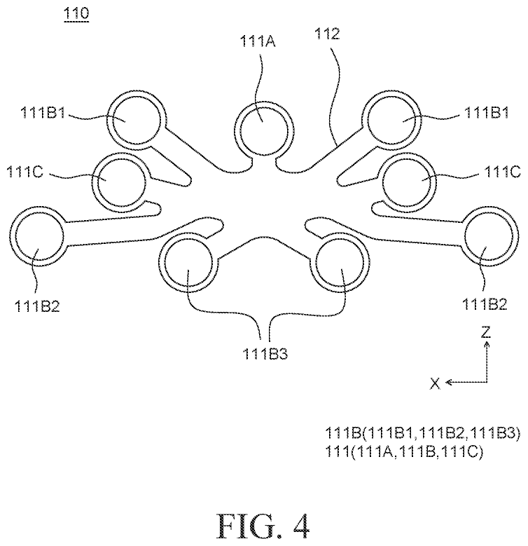

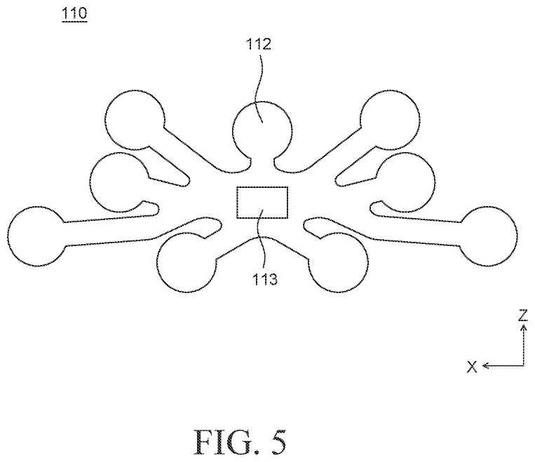

The sensor 110 will be described in more detail with reference to FIGS. 4 and 5.

FIG. 4 is a front view illustrating a sensor of the biosignal sensing device according to the embodiment of the present invention, and FIG. 5 is a rear view illustrating the sensor of the biosignal sensing device according to the embodiment of the present invention.

Referring to FIGS. 4 and 5, the sensor 110 may include a plurality of electrodes 111, a flexible printed circuit board 112, and a connection terminal 113.

The plurality of electrodes 111 may detect at least one biosignal while being in contact with the skin of the user U.

Specifically, the plurality of electrodes 111 may include a reference electrode 111A, an active electrodes 111B, and a ground electrodes 111C. For example, the plurality of electrodes 111 may be disposed to be spaced apart from one another according to International 10-to-20 electrode system.

The reference electrode 111A may be disposed at a reference point at signal measurement. Specifically, the reference electrode 111A may be disposed at a position where any biosignal is not detected, i.e., at a middle point of the sensor 110 where deflection does not occur and an influence of a jaw muscle is minimally exerted, that minimizes the occurrence of noise. Alternatively, the reference electrode 111A may be disposed at a position serving as a reference for disposition of the active electrodes 111B. For example, the active electrodes 111B may be disposed above and below the reference electrode 111A based on the reference electrode 111A.

The active electrodes 111B may be disposed in a horizontal direction with respect to the reference electrode 111A. That is, the active electrodes 111B may be disposed to face one side and the other side of the reference electrode 111A along an X-axis direction.

The active electrodes 111B may be disposed at a plurality of positions where neural activities are assumed to actually occur. The signals detected by the active electrodes 111B may be expressed by + or − values based on the signal of the reference electrode 111A. That is, a difference between the signal values may be measured depending on a difference between the positions of the active electrodes 111B and the position of the reference electrode 111A. Therefore, the position of the reference electrode 111A and the positions of the active electrodes 111B are considered important.

Specifically, the active electrodes 111B may include a plurality of active electrodes 111B1, 111B2, and 111B3 disposed at different positions based on a vertical direction (Z-axis direction). The plurality of active electrodes 111B1, 111B2, and 111B3 may include a first active electrode 111B1, a second active electrode 111B2, and a third active electrode 111B3. The plurality of active electrodes 111B1, 111B2, and 111B3 may each be provided as a pair of active electrodes. The plurality of active electrodes 111B1, 111B2, and 111B3 may be disposed at one side and the other side of the reference electrode 111A based on the horizontal direction, i.e., the X-axis direction. For example, the pair of first active electrodes 111B1 may be disposed above the reference electrode 111A based on the vertical direction, i.e., the Z-axis direction. And the pair of second active electrodes 111B2 may be disposed below the reference electrode 111A based on the vertical direction. Further, the pair of third active electrodes 111B3 may be disposed below the second active electrodes 111B2 based on the vertical direction. In the embodiment of the present invention, the configuration has been described in which the plurality of active electrodes 111B1, 111B2, and 111B3 includes the first active electrodes 111B1, the second active electrodes 111B2, and the third active electrodes 111B3. However, the present invention is not necessarily limited thereto, and more active electrodes may be further provided.

The ground electrodes 111C may be disposed in a horizontal direction with respect to the reference electrode 111A. That is, the ground electrodes 111C may be disposed to face one side and the other side of the reference electrode 111A along the X-axis direction. Further, the ground electrodes 111C may be disposed between the first active electrodes 111B1 and the second active electrodes 111B2 based on the vertical direction, i.e., the Z-axis direction. However, the position of the ground electrodes 111C is not necessarily limited thereto. And the position of the ground electrodes 111C may be changed to various positions. For example, the ground electrodes 111C may be attached to any position on the user's body and do not affect signal values of the reference electrode 111A or signal values of the active electrodes 111B.

The flexible printed circuit board 112 may be electrically connected to the plurality of electrodes 111 and the connection terminal 113 to transmit biosignals detected by the plurality of electrodes 111 to the connection terminal 113.

In addition, the flexible printed circuit board 112 may have a structure having flexibility so that the flexible printed circuit board 112 may be deformed to correspond to the shape of the contact portion CP. For example, the flexible printed circuit board 112 may include: a base film including a polyimide layer and a copper foil layer stacked on the polyimide layer; and a cover layer stacked on an upper surface of the base film. In addition, the flexible printed circuit board 112 may further include an insulating layer. The insulating layer surrounds an outer surface of the base film and an outer surface of the cover layer so that the electrodes 111 being in contact with the skin of the user U are partially exposed to the outside. The insulating layer may be made of a material having flexibility so that the insulating layer is bent in a shape corresponding to the contact portion CP. For example, the insulator may be made of various materials such as carbon or epoxy. In addition, an edge of the insulator may be sealed to prevent peeling phenomenon.

The connection terminal 113 may be disposed on a rear surface of the flexible printed circuit board 112 to electrically connected to the flexible printed circuit board 112 electrically connected to the plurality of electrodes 111. Further, the connection terminal 113 may be electrically connected to the control unit 140 configured to process the biosignals. The connection terminal 113 may transmit the biosignals detected by the plurality of electrodes 111 to the control unit 140. For example, the connection terminal 113 may include a terminal part physically connected to the control unit 140 by means of a cable, and a communication part connected to the control unit 140 in a wireless communication manner. Therefore, the connection terminal 113 and the control unit 140 may be connected to each other by means of any one of wired and wireless methods.

That is, the flexibility of the flexible printed circuit board 112 may allow the sensor 110 of the biosignal sensing device 100 according to the embodiment of the present invention to be applied various head shapes and to come into complete contact with the scalp. In addition, in the sensor 110 of the biosignal sensing device 100 according to the embodiment of the present invention, the plurality of electrodes 111, that detects the biosignals at the plurality of positions, may cover the entire region of the prefrontal lobe to acquire the biosignals in the entire region of the prefrontal lobe. In addition, the plurality of electrodes 111 is formed to be very thin, such that the user cannot feel contact with the plurality of electrodes 111 when the plurality of electrodes 111 is in contact with the skin. When the plurality of electrodes 111 comes into contact with the contact portion CP, the plurality of electrodes 111 may be deformed to a shape corresponding to the contact portion CP, such that the plurality of electrodes 111 may be in complete contact with the contact portion CP. Therefore, it is possible to accurately measure the biosignals.

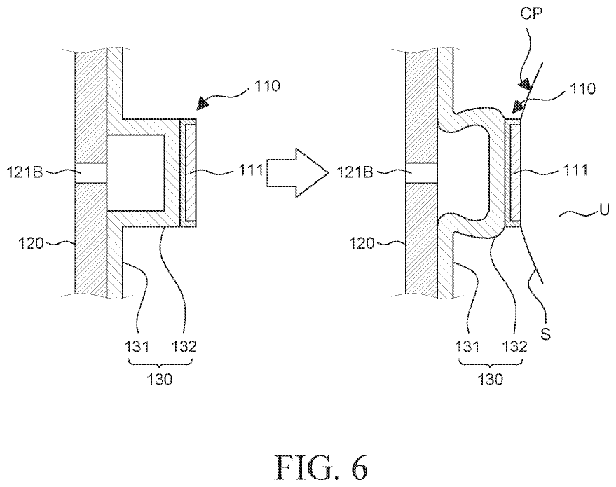

FIG. 6 is a view schematically illustrating a change of a pressing protrusion when a support pad of the biosignal sensing device according to the embodiment of the present invention brings the sensor into close contact with a user's skin.

Referring to FIGS. 1, 3, and 6, the biosignal sensing device 100 may further include a bracket 120 and a support pad 130.

The bracket 120 may be coupled to the head-mounted display device HMD.

And the bracket 120 may be coupled to the support pad 130 having one surface to which the sensor 110 is coupled, such that the support pad 130 may be disposed to face the contact portion CP. For example, the bracket 120 may be made of a plastic material so as to have predetermined hardness and elasticity. However, the bracket 120 is not necessarily limited thereto, and may be made of various materials.

The bracket 120 may include a support plate 121 and a coupling member 122.

The support plate 121 may be coupled to the support pad 130. The support plate 121 may be connected to the coupling member 122 coupled to the head-mounted display device HMD. The support plate 121 may be disposed to face the face of the user U, who wears the head-mounted display device HMD, and elastically supported on the coupling member 122. Therefore, when the user U wears the head-mounted display device HMD, the support plate 121 elastically supported on the coupling member 122 may press the support pad 130 toward the contact portion CP.

The support plate 121 may have a shape curved in the horizontal direction, i.e., the X-axis direction. Therefore, the support plate 121 may be applied to various head shapes. However, the support plate 121 is not necessarily limited thereto. The support plate 121 may be formed in a shape curved in the horizontal direction and the vertical direction (Z-axis direction).

The support plate 121 may include a through-hole 121A and a plurality of air discharge holes 121B.

The through-hole 121A may be disposed at a center of the support plate 121 to expose the connection terminal 113 of the sensor 110 penetrating the support pad 130 to an external space.

The air discharge hole 121B may be formed at positions corresponding to pressing protrusions 132 disposed on the support pad 130. Accordingly, the air discharge holes 121B may prevent an air layer from being formed between the support plate 121 and the pressing protrusions 132 configured to elastically support the electrodes 111. That is, when the pressing protrusion 132, that brings the electrode 111 into close contact with the skin S of the user U, is compressed by being pushed by the skin S being in close contact with the electrode 111, air between the pressing protrusion 132 and the support plate 121 is discharged to the outside through the air discharge holes 121B. Therefore, the air layer, that hinders the movement of the pressing protrusion 132, is removed from the portion between the pressing protrusion 132 and the support plate 121. Therefore, the pressing protrusions 132 pressed against the skin S of the user U may be naturally compressed, and the pressure applied to the skin of the user U may be minimized.

The coupling member 122 may extend from an upper end of the support plate 121 and be coupled to the head-mounted display device HMD. The coupling member 122 may elastically support the support plate 121. For example, the coupling member 122 may have a coupling portion capable of being coupled to the head-mounted display device HMD. The coupling portion may have a structure capable of being detachably mounted on the head-mounted display device HMD.

The coupling member 122 may extend in an arcuate shape from the upper end of the support plate 121 and elastically support the support plate 121. That is, when the user U wears the head-mounted display device HMD, the coupling member 122 is pressed by the support plate 121. In this case, since the coupling member 122 is formed in an arcuate shape at the upper end of the support plate 121, the coupling member 122 may absorb a load applied from the support plate 121 while being compressed by a magnitude of the load applied from the support plate 121. Further, the coupling member 122 may bring the electrode 111 of the sensor 110 into complete contact with the skin S of the user U by pressing the support plate 121 toward the contact portion CP by applying the elastic force.

In the embodiment of the present invention, the configuration has been described that the bracket 120 of the biosignal sensing device 100 includes the support plate 121 and the coupling member 122. However, the bracket 120 is not necessarily limited thereto. The bracket 120 may be added or modified depending on a shape of the head-mounted display device HMD to which the biosignal sensing device 100 is to be coupled. In addition, the bracket 120 may be separated from the head-mounted display device HMD and configured to be independently worn by the user.

Referring to FIGS. 1 and 6, the support pad 130 may be coupled to the support plate 121 of the bracket 120 so that the sensor 110 coupled to one side of the support pad 130 may face the contact portion CP. Further, when the user U wears the head-mounted display device HMD, the support pad 130 is pressed by the bracket 120 that applies a reaction force toward the contact portion CP, such that the sensor 110 coupled to one side of the support pad 130 may come into close contact with the skin S of the user U.

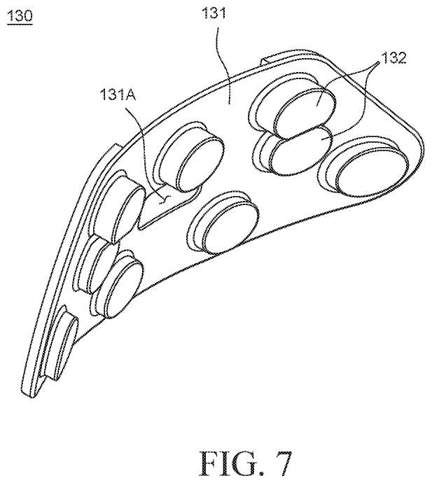

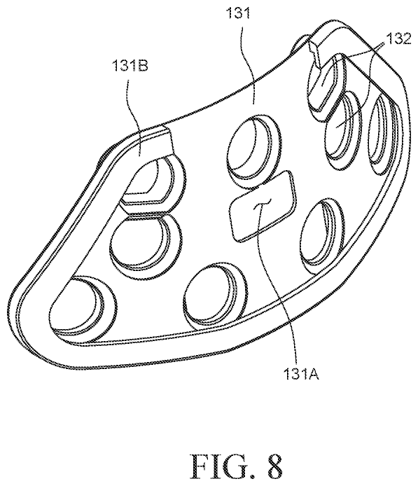

FIG. 7 is a perspective view illustrating a state of the support pad of the biosignal sensing device according to the embodiment of the present invention when viewed from the front side, and FIG. 8 is a perspective view illustrating a state of the support pad of the biosignal sensing device according to the embodiment of the present invention when viewed from the rear side.

Referring to FIGS. 6 to 8, the support pad 130 may be made of a material having elasticity.

Specifically, the support pad 130 may be made of silicone that is easily deformed in shape. Therefore, the support pad 130 may maintain a shape corresponding to an external shape of the bracket 120 when the support pad 130 is coupled to the bracket 120. Further, when the user U wears the head-mounted display device HMD, the support pad 130 may be deformed to a shape corresponding to the contact portion CP so that the sensor 110 coupled to one surface of the support pad 130 may come into close contact with the contact portion CP.

That is, when the user U wears the head-mounted display device HMD, the electrode 111 provided in the biosignal sensing device 100 needs to be kept in close contact with the contact portion CP. And at the same time, the electrode 111 needs to maintain a state that the force pressing the contact portion CP is minimized. Therefore, in the biosignal sensing device 100 according to the embodiment of the present invention, the support pad 130, which brings the sensor 110 coupled to one surface of the support pad 130 into close contact with the contact portion CP when the user U wears the head-mounted display device HMD, is made of silicone. Therefore, it is possible to bring the electrode 111 into complete contact with the contact portion CP. And it is possible to minimize the force applied by the electrode 111 to press the contact portion CP.

In addition, the support pad 130 may have a concave-convex structure that elastically supports the plurality of electrodes 111.

Specifically, the support pad 130 may include: a base 131 coupled to the bracket 120 and configured to maintain a shape corresponding to the bracket 120; and a plurality of pressing protrusions 132 provided on the base 131.

Referring to FIGS. 3, 5, and 8, the base 131 may have a shape corresponding to the support plate 121 of the bracket 120. Further, the base 131 may have a coupling portion 131B that may be coupled to the edge of the support plate 121. In addition, a through-hole 131A may be formed at a center of the base 131, and the connection terminal 113 of the sensor 110 may be disposed in the through-hole 131A.

Referring to FIGS. 3, 6, and 7, the plurality of pressing protrusions 132 may be disposed at positions corresponding to the plurality of air discharge holes 121B formed in the support plate 121. And the plurality of electrodes 111 disposed on one surface through the plurality of pressing protrusions 132 may be disposed at a position spaced apart from one surface of the base 131 at a predetermined distance. That is, when the user U wears the head-mounted display device HMD, the plurality of pressing protrusions 132 may bring all the plurality of electrodes 111, spaced apart from one surface of the base 131, into close contact with the skin of the user U.

The biosignal sensing device 100 according to the embodiment of the present invention has the concave-convex structure applied to the support pad 130 that brings the sensor 110 into contact with the skin S of the user U. Therefore, even though a shape or size of the head of the user U changes, the support pad 130 may bring the electrode 111 into completely close contact with the skin S of the user U, thereby maintaining the state in which the electrode 111 and the skin S are in contact with one another. Therefore, the quality of the detected biosignal may be maintained in the highest state.

Referring to FIGS. 3, 6, and 8, the plurality of pressing protrusions 132 may elastically support the plurality of electrodes 111. In this case, the plurality of pressing protrusions 132 may each have a shell structure having a vacant internal space.

Therefore, when the user U wears the head-mounted display device HMD, the plurality of pressing protrusions 132, that supports the plurality of electrodes 111, may be pressed and compressed by the contact portion CP. Further, the plurality of compressed pressing protrusions 132 may elastically support the plurality of electrodes 111 toward the contact portion CP, thereby bringing the plurality of electrodes 111 into completely close contact with the skin S of the user U.

That is, since the plurality of pressing protrusions 132, that supports the plurality of electrodes 111, has the shell structure having a vacant internal space, an external shape of each of the plurality of pressing protrusions 132 is deformed by being pressed by the contact portion CP when the user U wears the head-mounted display device HMD. Therefore, it is possible to minimize the force applied to press the skin S of the user U and minimize fatigue caused by the pressing. In addition, since the plurality of pressing protrusions 132 are compressed when the external shape is deformed to elastically support the plurality of electrodes 111, such that the plurality of electrodes 111 may be in completely close contact with the skin S of the user U.

Referring to FIG. 1, the biosignal sensing device 100 may further include the control unit 140.

The control unit 140 may be electrically connected to the sensor 110, and receive at least one biosignal detected by the sensor 110. The control unit 140 may process and convert the received biosignal and transmit the biosignal to an external device.

The control unit 140 may amplify and process at least one biosignal detected by the sensor 110, perform A/D conversion on the processed biosignal, and transmit the biosignal to the external device connected to the control unit 140 in a wired or wireless communication manner. For example, the external devices may include a user terminal, the head-mounted display device, a server, and the like.

Specifically, the control unit 140 may include: a amplifying part configured to amplify the biosignal detected by the sensor 110; a signal processing part configured to remove noise from the amplified signal; a power supplying part configured to supply power; an A/D conversion part configured to convert the nosie-removed biosignal into a digital signal; a communication part connected to the external device in a wired or wireless manner and configured to transmit the biosignal converted into the digital signal to the external device; and a controller configured to control the above-mentioned components.

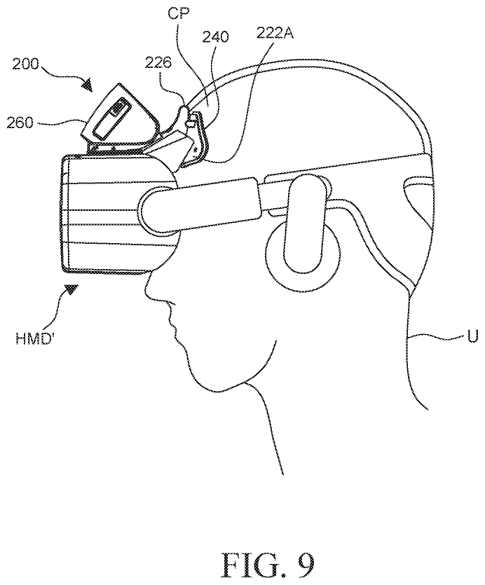

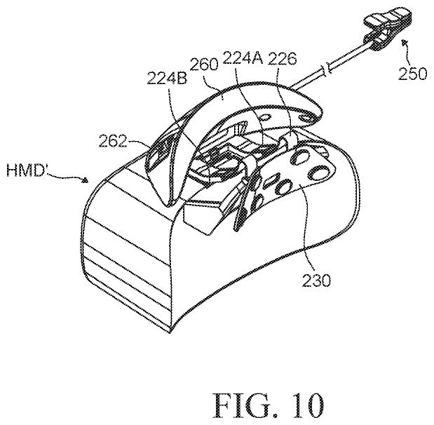

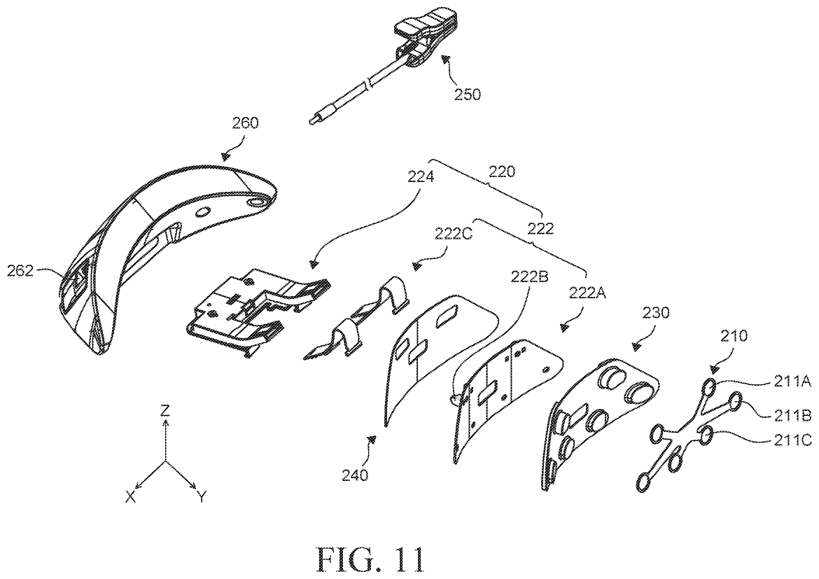

FIG. 9 is a view schematically illustrating a state in which a user wears a head-mounted display device to which a biosignal sensing device according to another embodiment of the present invention is coupled, FIG. 10 is a rear perspective view illustrating the head-mounted display device to which the biosignal sensing device according to another embodiment of the present invention is coupled, and FIG. 11 is an exploded perspective view illustrating the biosignal sensing device according to another embodiment of the present invention.

Referring to FIGS. 9 to 11, a biosignal sensing device 200 according to another embodiment of the present invention may be detachably mounted on a head-mounted display device HMD′. And the biosignal sensing device 200 detect a biosignal while being in contact with skin of the user who wears the head-mounted display device HMD′.

Specifically, the biosignal sensing device 200 coupled to the head-mounted display device HMD′ may be disposed at an upper side of the head-mounted display device HMD. when the user U wears the head-mounted display device HMD′, the biosignal sensing device 200 may detect at least one biosignal while being in contact with a forehead portion and ears of a user U.

The head-mounted display device HMD′ may have a structure that can be worn on the head of the user U. Further, the head-mounted display device HMD′ may provide the user U with virtual reality (VR) images to enable the user U to have a spatial, temporal experience similar to an actual experience.

In the present embodiment, the configuration has been described in which the biosignal sensing device 200 has the structure detachably mounted on the head-mounted display device HMD′. However, the biosignal sensing device 200 according to the embodiment of the present invention may not be necessarily detachably mounted only on the head-mounted display device HMD′. The biosignal sensing device 200 may be formed in structure that can be used independently while not coupled to the head-mounted display device HMD′.

Referring to FIGS. 9 to 11, the biosignal sensing device 200 may include a sensor 210.

The sensor 210 is supported by a support pad 230 coupled to a bracket 220. The sensor 210 detects at least one biosignal while being disposed on the forehead portion of the user U, who wears the head-mounted display device HMD′, and being in contact with the skin of the user U.

Specifically, the sensor 210 may measure a brainwave signal of a prefrontal lobe while being in contact with the forehead portion of the user U. However, the sensor 210 may not necessarily measure only the brainwave signal of the prefrontal lobe while being in contact with the forehead portion of the user U. The sensor 210 may be configured to detect various biosignals while being in contact with various body portions of the user U.

In addition, when the sensor 210 is in contact with the body of the user U, the sensor 210 may be deformed to a shape corresponding to a contact portion CP. And the sensor 210 may be in complete contact with the contact portion CP.

Specifically, the sensor 210 may be provided in the form of a film having flexibility so that the sensor 210 may be bent or curved and thus be deformed to a shape corresponding to the contact portion CP. Therefore, even though a shape of the contact portion CP is changed, the sensor 210 may be deformed to a shape corresponding to the contact portion CP, such that the sensor 210 may always maintain in state being completely close contact with the contact portion CP.

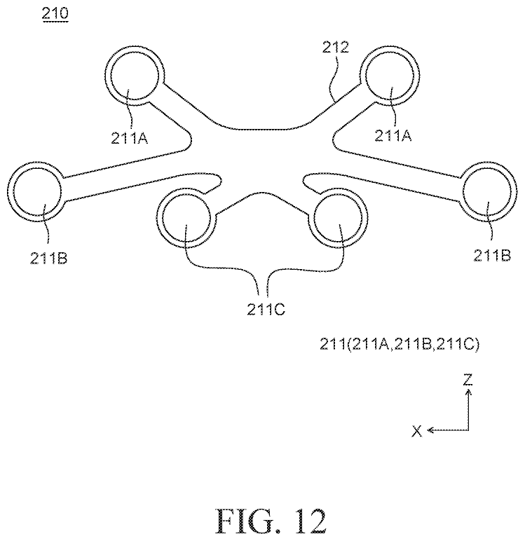

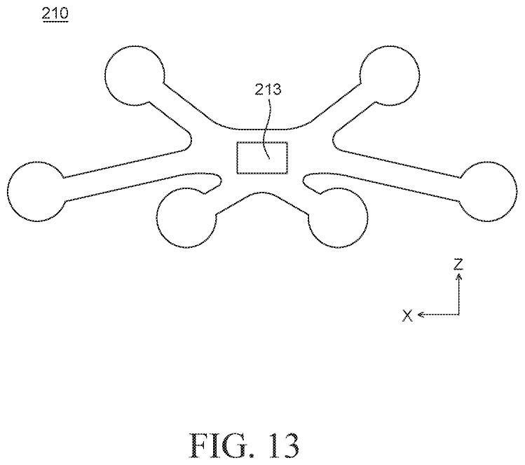

The sensor 210 will be described in more detail with reference to FIGS. 12 and 13.

FIG. 12 is a front view illustrating a sensor of the biosignal sensing device according to another embodiment of the present invention, and FIG. 13 is a rear view illustrating the sensor of another biosignal sensing device according to another embodiment of the present invention.

Referring to FIGS. 12 and 13, the sensor 210 may include a plurality of active electrodes 211, a flexible printed circuit board 212, and a connection terminal 213. The plurality of active electrodes 211 may detect at least one biosignal while being in contact with the skin of the user U.

The plurality of active electrodes 211 may be disposed at one side and the other side of the connection terminal 213 based on the X-axis direction with respect to the connection terminal 213.

The plurality of active electrodes 211 may be disposed at a plurality of positions at which neural activities are assumed to actually occur. The signals detected by the respective active electrode 211 may be expressed by + or − values based on the signal of a reference electrode 253. That is, a difference between the signal values may be measured depending on a difference between the positions of the respective active electrode 211 and the position of the reference electrode 253. Therefore, the position of the reference electrode 253 and the positions of the plurality of active electrodes 211 are considered important.

The plurality of active electrodes 211 may include a first active electrodes 211A, a second active electrodes 211B, and a third active electrodes 211C. The plurality of active electrodes 211 may each be provided as a pair of active electrodes. The plurality of active electrodes 211 may be disposed at one side and the other side of the connection terminal 213 based on the X-axis direction. For example, the pair of first active electrodes 211A may be disposed above the connection terminal 213 based on the Z-axis direction, and the pair of second active electrodes 211B may be disposed below the connection terminal 213 based on the Z-axis direction. Further, the pair of third active electrodes 211C may be disposed below the second active electrodes 211B based on the Z-axis direction. In the embodiment of the present invention, the configuration has been described that the plurality of active electrodes 211 includes the first active electrodes 211A, the second active electrodes 211B, and the third active electrodes 211C. However, the present invention is not necessarily limited thereto, and more active electrodes may be further provided.

The flexible printed circuit board 212 may be electrically connected to the plurality of active electrodes 211 and the connection terminal 213 to transmit biosignals detected by the plurality of active electrodes 211 to the connection terminal 213.

In addition, the flexible printed circuit board 212 may have a structure having flexibility so that the flexible printed circuit board 212 may be deformed to correspond to the shape of the contact portion CP. For example, the flexible printed circuit board 212 may include: a base film including a polyimide layer and a copper foil layer stacked on the polyimide layer; and a cover layer stacked on an upper surface of the base film. In addition, the flexible printed circuit board 212 may further include an insulating layer. The insulating layer surrounds an outer surface of the base film and an outer surface of the cover layer so that the plurality of active electrodes 211 being in contact with the skin of the user U is partially exposed to the outside. The insulating layer may be made of a material having flexibility so that the insulating layer is bent in a shape corresponding to the contact portion CP. For example, the insulator may be made of various materials such as carbon or epoxy. In addition, an edge of the insulator may be sealed to prevent peeling phenomenon.

The connection terminal 213 may be disposed on a rear surface of the flexible printed circuit board 212 to electrically connected to the flexible printed circuit board 212 electrically connected to the plurality of active electrodes 211. Further, the connection terminal 213 may be electrically connected to a control unit 260 configured to process the biosignals. The connection terminal 213 may transmit the biosignals detected by the plurality of active electrodes 211 to the control unit 260. For example, the connection terminal 213 may include a terminal part physically connected to the control unit 260 by means of a cable, and a communication part connected to the control unit 260 in a wireless communication manner. Therefore, the connection terminal 213 and the control unit 260 may be connected to each other by means of any one of wired and wireless methods.

That is, the flexibility of the flexible printed circuit board 212 may allow the sensor 210 of the biosignal sensing device 200 according to the embodiment of the present invention to be applied various head shapes and to come into complete contact with the scalp. In addition, in the sensor 210 of the biosignal sensing device 200 according to the embodiment of the present invention, the plurality of active electrodes 211, that detects the biosignals at the plurality of positions, may cover the entire region of the prefrontal lobe to acquire the biosignals in the entire region of the prefrontal lobe. In addition, the plurality of active electrodes 211 is formed to be very thin, such that the user cannot feel contact with the plurality of active electrodes 211 when the plurality of active electrodes 211 is in contact with the skin. When the plurality of active electrodes 211 comes into contact with the contact portion CP, the plurality of active electrodes 211 may be deformed to a shape corresponding to the contact portion CP, such that the plurality of active electrodes 211 may be in complete contact with the contact portion CP. Therefore, it is possible to accurately measure the biosignals.

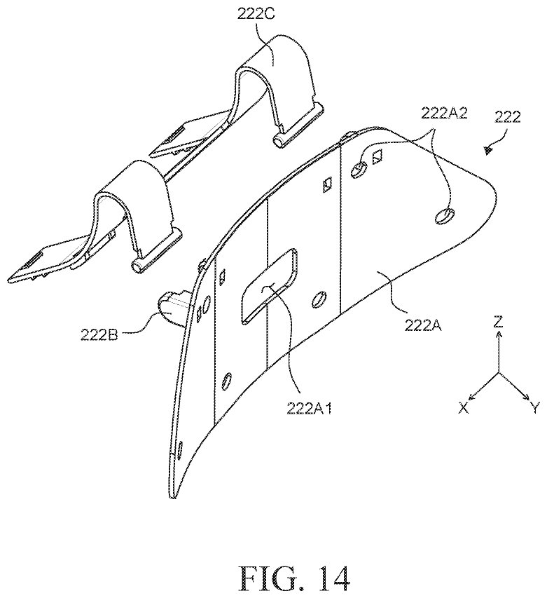

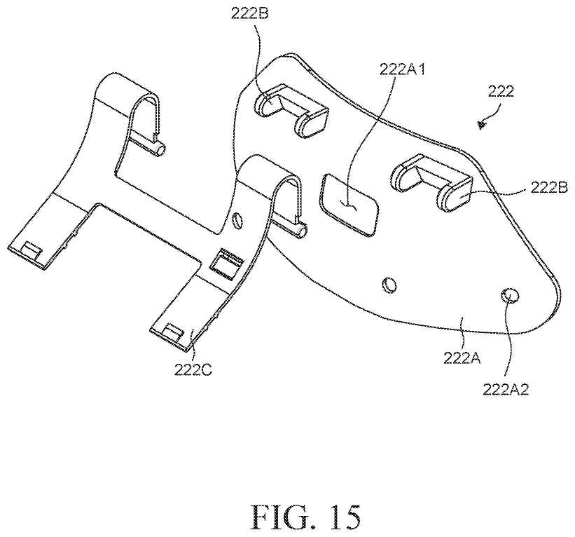

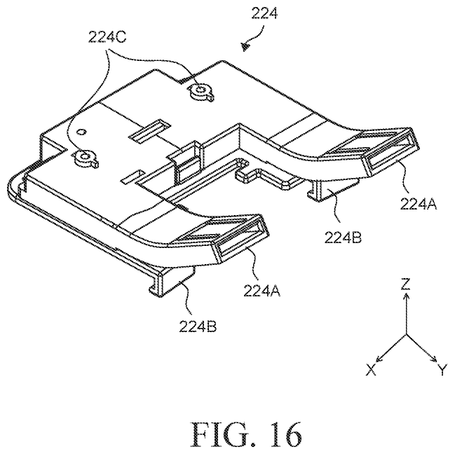

FIG. 14 is a perspective view illustrating a state in which a coupling member of a first bracket of the biosignal sensing device according to another embodiment of the present invention is separated from a support plate when viewed from the front side, FIG. 15 is a perspective view illustrating a state in which the coupling member of the first bracket of the biosignal sensing device according to another embodiment of the present invention is separated from the support plate when viewed from the rear side, and FIG. 16 is a perspective view illustrating a second bracket of a bracket of the biosignal sensing device according to another embodiment of the present invention.

Referring to FIGS. 11 and 14 to 16, the biosignal sensing device 200 may further include a bracket 220.

The bracket 220 may be coupled to the head-mounted display device HMD′ and the control unit 260. And the bracket 220 may coupled to the support pad 230 having one surface to which the sensor 210 is coupled, such that the support pad 230 may be disposed to face the contact portion CP. For example, the bracket 220 may be made of a plastic material so as to have predetermined hardness and elasticity. However, the bracket 220 is not necessarily limited thereto, and may be made of various materials.

The bracket 220 may include a first bracket 222 and a second bracket 224.

The first bracket 222 may include a support plate 222A, a hangers 222B, and a coupling member 222C.

The support plate 222A may be coupled to the support pad 230. The support plate 222A may be connected to the coupling member 222C and disposed to face the face of the user U who wears the head-mounted display device HMD′. The support plate 222A may be elastically supported on the coupling member 222C coupled to the hangers 222B. Therefore, when the user U wears the head-mounted display device HMD′, the support plate 222A elastically supported on the coupling member 222C may press the support pad 230 toward the contact portion CP.

The support plate 222A may have a shape curved in the X-axis direction. Therefore, the support plate 222A may be applied to various head shapes. However, the support plate 222A is not necessarily limited thereto. The support plate 222A may be formed in a shape curved in the Z-axis direction.

The support plate 222A may include a through-hole 222A1 and a plurality of first air discharge holes 222A2.

The through-hole 222A1 may be disposed at a center of the support plate 222A. And the connection terminal 213 of the sensor 210, that penetrates the support pad 230, may be exposed to an external space through the through-hole 222A1.

The first air discharge hole 222A2 may be formed at positions corresponding to pressing protrusions 232 disposed on the support pad 230. And The first air discharge hole 222A2 may prevent an air layer from being formed between the support plate 222A and the plurality of pressing protrusions 232 configured to elastically support the plurality of active electrodes 211.

That is, when the plurality of pressing protrusions 232, that brings the plurality of active electrodes 211 into close contact with the skin S of the user U, is compressed by being pushed by the skin S being in close contact with the plurality of active electrodes 211, air between the plurality of pressing protrusions 232 and the support plate 222A is discharged through the first air discharge hole 222A2. And then the discharged air may be discharged to the outside through a second air discharge hole H formed between a coupling portion 231B of the support pad 230 and a buffer pad 240.

Therefore, the air layer, that hinders the movements of the plurality of pressing protrusions 232, is removed from the portion between the plurality of pressing protrusions 232 and the support plate 222A. Therefore, the plurality of pressing protrusions 232 pressed against the skin S of the user U may be naturally compressed, and the pressure applied to the skin of the user U may be minimized.

The hangers 222B may be disposed on one surface of the support plate 222A to accommodate the coupling member 222C so that the coupling member 222C is rotatable.

The first bracket 222 may be rotated by the coupling member 222C coupled to the hangers 222B, and the hangers 222B may finely rotate the support plate 222A pressed toward the contact portion CP. Therefore, the hangers 222B may rotate the support plate 222A and assist the support plate 222A so that the support pad 230 may come into contact with any portion of the of the forehead of the user U.

The coupling member 222C may be rotatably coupled to the hangers 222B of the first bracket 222, and coupled to a first coupling portion 224A of the second bracket 224.

One end of the coupling member 222C may be formed in an arcuate shape and elastically support the support plate 222A. That is, when the user U wears the head-mounted display device HMD′, the coupling member 222C is pressed by the support plate 222A. Since one end of the coupling member 222C is formed in an arcuate shape, the coupling member 222C may absorb a load applied from the support plate 222A while being compressed by a magnitude of the load applied from the support plate 222A. Further, the coupling member 222C may bring the plurality of active electrodes 211 of the sensor 210 into completely close contact with the skin S of the user U by pressing the support plate 222A toward the contact portion CP by applying the elastic force.

The second bracket 224 may be disposed between the head-mounted display device HMD′ and the control unit 260. The second bracket 224 may fix the coupling member 222C coupled to the support plate 222A. In addition, the second bracket 224 may fix the control unit 260 so that the control unit 260 may not be swayed even by external impact.

The second bracket 224 may include the first coupling portion 224A, a second coupling portion 224B, and a third coupling portion 224C.

The first coupling portion 224A may be coupled to the coupling member 222C. The first coupling portion 224A is inclined by a predetermined angle along a gradient of a shape of one end of the coupling member 222C. The coupling member 222C may moved while being guided inside the first coupling portion 224A inclined by a predetermined angle.

The second coupling portion 224B may be coupled to the head-mounted display device HMD′. The second coupling portion 224B may be provided in the form of a hook and fix the second bracket 224 to the head-mounted display device HMD′.

The third coupling portion 224C may fix the control unit 260. Therefore, even when the user U rotates in the left or right direction around Z-axis direction while wearing the head-mounted display device HMD′, the control unit 260 may not be separated from the head-mounted display device HMD′.

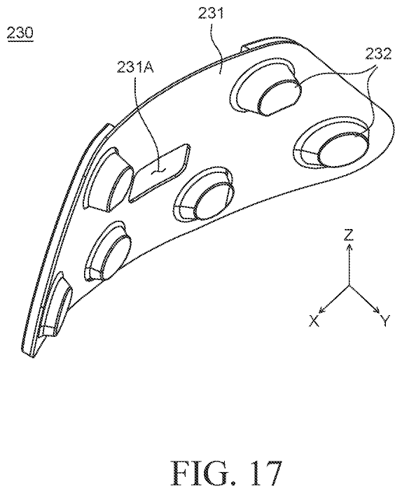

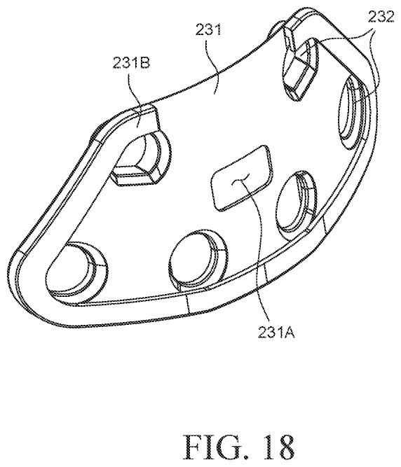

FIG. 17 is a perspective view illustrating a state of the support pad of the biosignal sensing device according to another embodiment of the present invention when viewed from the front side, and FIG. 18 is a perspective view illustrating a state of the support pad of the biosignal sensing device according to another embodiment of the present invention when viewed from the rear side.

Referring to FIGS. 17 and 18, the support pad 230 may be coupled to the buffer pad 240 so that the sensor 210 coupled to one side of the support pad 230 may be disposed to face the contact portion CP. Further, when the user U wears the head-mounted display device HMD′, the support pad 230 is pressed by the first bracket 222 that applies a reaction force toward the contact portion CP, such that the sensor 210 coupled to one side of the support pad 230 may come into close contact with the skin S of the user U.

The support pad 230 may be made of a material having elasticity.

Specifically, the support pad 230 may be made of silicone that is easily deformed in shape. Therefore, the support pad 230 may maintain a shape corresponding to an external shape of the support plate 222A when the support pad 230 is coupled to the first bracket 222. Further, when the user U wears the head-mounted display device HMD′, the support pad 230 may be deformed to a shape corresponding to the contact portion CP so that the sensor 210 coupled to one surface of the support pad 230 may come into close contact with the contact portion CP.

That is, when the user U wears the head-mounted display device HMD′, the plurality of active electrodes 211 provided in the biosignal sensing device 200 needs to be kept in close contact with the contact portion CP. And at the same time, the plurality of active electrodes 211 needs to maintain a state that the force pressing the contact portion CP is minimized. Therefore, in the biosignal sensing device 200 according to the embodiment of the present invention, the support pad 230, which brings the sensor 210 coupled to one surface of the support pad 230 into close contact with the contact portion CP when the user U wears the head-mounted display device HMD, is made of silicone. Therefore, it is possible to bring the plurality of active electrodes 211 into complete contact with the contact portion CP. And it is possible to minimize the force applied by the plurality of active electrodes 211 to press the contact portion CP.

In addition, the support pad 230 may have a concave-convex structure that elastically supports the plurality of active electrodes 211.

Specifically, the support pad 230 may include: a base 231 coupled to the first bracket 222 and configured to maintain a shape corresponding to the buffer pad 240; and a plurality of pressing protrusions 232 provided on the base 231.

The base 231 may have a shape corresponding to the support plate 222A of the first bracket 222. Further, the base 231 may have the coupling portion 231B that may be coupled to the edge of the support plate 222A and the edge of the buffer pad 240. In addition, a through-hole 231A may be formed at a center of the base 231, and the connection terminal 213 of the sensor 210 may be disposed in the through-hole 231A.

The plurality of pressing protrusions 232 may be disposed at positions corresponding to the plurality of first air discharge holes 222A2 formed in the first bracket 222. And the plurality of active electrodes 211 disposed on one surface through the plurality of pressing protrusions 232 may be disposed at a position spaced apart from one surface of the base 231 at a predetermined distance. That is, when the user U wears the head-mounted display device HMD′, the plurality of pressing protrusions 232 may bring all the plurality of active electrodes 211, spaced apart from one surface of the base 231, into close contact with the skin of the user U.

The biosignal sensing device 200 according to another embodiment of the present invention has the concave-convex structure applied to the support pad 230 that brings the sensor 210 into contact with the skin S of the user U. Therefore, even though a shape or size of the head of the user U changes, the support pad 230 may bring the plurality of active electrodes 211 into completely close contact with the skin S of the user U, thereby maintaining the state in which the plurality of active electrodes 211 and the skin S are in contact with one another. Therefore, the quality of the detected biosignal may be maintained in the highest state.

The plurality of pressing protrusions 232 may elastically support the plurality of active electrodes 211. In this case, the plurality of pressing protrusions 232 may each have a shell structure having a vacant internal space.

That is, since the plurality of pressing protrusions 232, that supports the plurality of active electrodes 211, has the shell structure having a vacant internal space, an external shape of each of the plurality of pressing protrusions 232 is deformed by being pressed by the contact portion CP when the user U wears the head-mounted display device HMD′. Therefore, it is possible to minimize the force applied to press the skin S of the user U and minimize fatigue caused by the pressing. In addition, since the plurality of pressing protrusions 232 are compressed when the external shape is deformed to elastically supports the plurality of active electrodes 211, such that the plurality of active electrodes 211 may be in completely close contact with the skin S of the user U.

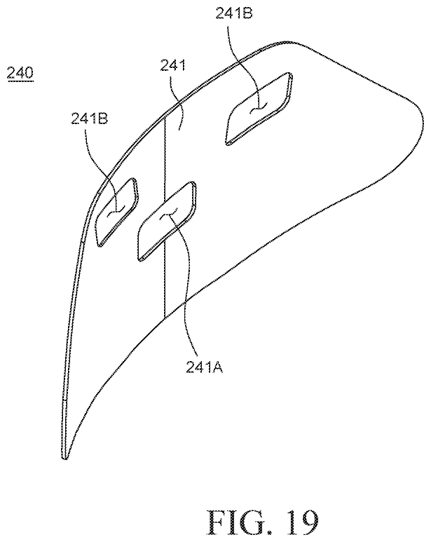

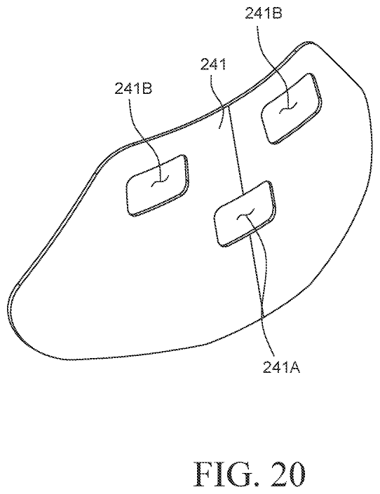

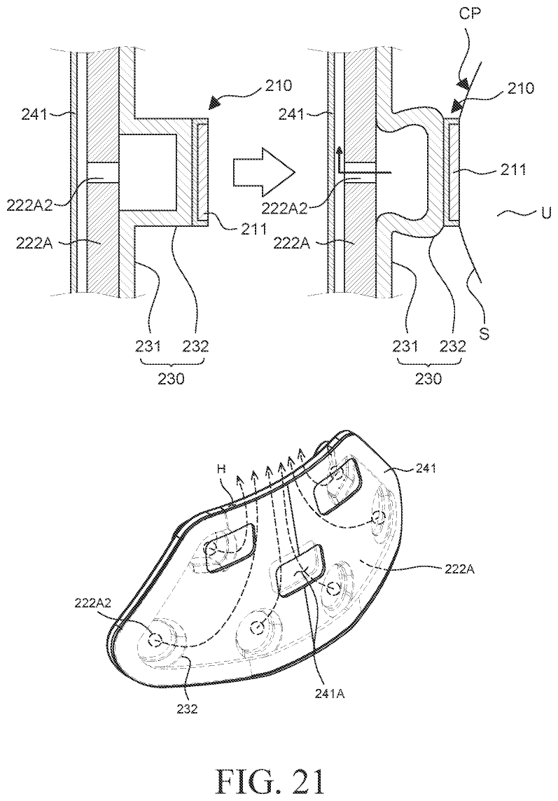

FIG. 19 is a perspective view illustrating a state of the buffer pad of the biosignal sensing device according to another embodiment of the present invention when viewed from the front side, FIG. 20 is a view perspective view illustrating a state of the buffer pad of the biosignal sensing device according to another embodiment of the present invention when viewed from the rear side, and FIG. 21 is a view schematically illustrating changes of the pressing protrusion and a buffer protrusion when the support pad of the biosignal sensing device according to another embodiment of the present invention brings the sensor into contact with the user's skin.

Referring to FIGS. 19 to 20, the buffer pad 240 may be coupled to the outer surface of the coupling portion 231B of the support pad 230. The buffer pad 240 may have a shape curved in the X-axis direction. Therefore, the buffer pad 240 may be coupled to the outer surface of the coupling portion 231B of the support pad 230. However, the buffer pad 240 is not necessarily limited thereto, and may be formed in a shape curved in the Z-axis direction.

When the user U wears the head-mounted display device HMD′, the buffer pad 240 may minimize the pushing of the support pad 230 that receives a pushing force from the contact portion CP.

The buffer pad 240 may be made of a material having elasticity.

Specifically, the buffer pad 240 may be made of silicone that is easily deformed in shape. Therefore, the buffer pad 240 may maintain a shape corresponding to an external shape of the support pad 230 so as to cover the coupling portion 231B of the support pad 230 when the buffer pad 240 is coupled to the support pad 230. Further, when the user U wears the head-mounted display device HMD′, the buffer pad 240 is deformed to a shape corresponding to the contact portion CP together with the support pad 230 so that the sensor 210 coupled to one surface of the buffer pad 240 may come into close contact with the contact portion CP.

The buffer pad 240 may include a buffer plate 241 coupled to the support pad 230 and configured to maintain a shape corresponding to the support pad 230.

A through-hole 241A may be formed at a center of the buffer plate 241, and the connection terminal 213 of the sensor 210 may be disposed in the through-hole 241A. The through-hole 241A may communicate with the through-hole 231A of the support pad 230.

In addition, the buffer plate 241 may have hanger accommodation hole 241B configured to accommodate the hangers 222B of the first bracket 222. The hanger accommodation hole 241B may be disposed above the through-hole 241A. The plurality of hanger accommodation hole 241B may be disposed to be spaced apart from one another with respect to the through-hole 241A.

Referring to FIG. 21, the plurality of pressing protrusions 232 may be disposed at the positions corresponding to the plurality of first air discharge holes 222A2 formed in the support plate 222A. Therefore, when the user U wears the head-mounted display device HMD′, the plurality of pressing protrusions 232 may bring all the plurality of active electrodes 211 into close contact with the skin of the user U.

The plurality of pressing protrusions 232 may elastically support the plurality of active electrodes 211. In this case, the plurality of pressing protrusions 232 may each have a shell structure having a vacant internal space.

Therefore, when the user U wears the head-mounted display device HMD′, the plurality of pressing protrusions 232, that supports the plurality of active electrodes 211, may be pressed and compressed by the contact portion CP. Further, the plurality of compressed pressing protrusions 232 may elastically support the plurality of active electrodes 211 toward the contact portion CP, thereby bringing the plurality of active electrodes 211 into completely close contact with the skin S of the user U.

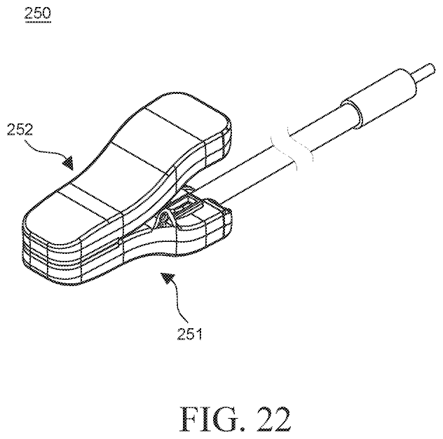

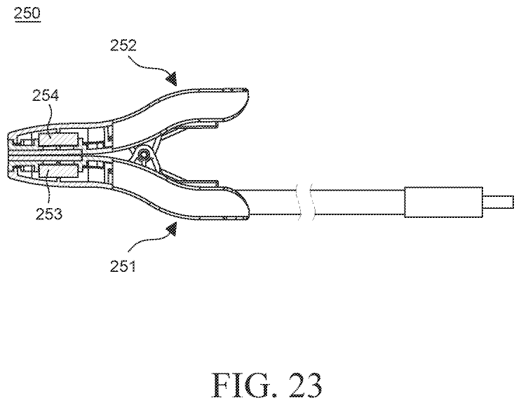

FIG. 22 is a perspective view a state of an ear clip of the biosignal sensing device according to another embodiment of the present invention when viewed from the front side, and FIG. 23 is a view illustrating a cross-section of a part of the ear clip of the biosignal sensing device according to another embodiment of the present invention.

Referring to FIGS. 22 and 23, the biosignal sensing device 200 may further include an ear clip 250.

The ear clip 250 may be coupled to the ear of the user U and configured to generate a potential difference with the plurality of active electrodes 211.

The ear clip 250 may include a first body 251, a second body 252, a reference electrode 253, and a ground electrode 254.

The first body 251 may come into contact with one surface of the user's ear lobe, And the first body 251 may have a space that may accommodate the reference electrode 253 therein. An accommodation hole may be formed at one side of the first body 251 and a connection cable may be accommodated therein. The accommodation hole is connected to the connection cable, and a signal of the reference electrode 253 may be transmitted to the control unit 260 through the connection cable.

The second body 252 may come into contact with the other surface of the user's ear lobe, that is opposite to one surface of the user's ear lobe. And the second body 252 may have a space that may accommodate the ground electrode 254 therein. The first body 251 and the second body 252 are connected to each other by means of a spring. As the first body 251 and the second body 252 are spread outward and retracted inward with respect to the spring, the first body 251 and the second body 252 may be fixed to the ear lobe of the user U.

The reference electrode 253 may be accommodated in the first body 251. The reference electrode 253 may be a criterion for signal measurement. That is, a difference between the signal values may be measured depending on a difference between the position of the reference electrode 253 and the positions of the plurality of active electrodes 211.

The ground electrode 254 may be accommodated in the second body 252. The ground electrode 254 does not affect the signal values of the reference electrode 253 or the signal values of the plurality of active electrodes 211.

In the present embodiment, the reference electrode 253 is accommodated in the first body 251, and the ground electrode 254 is accommodated in the second body 252. However, the present invention is not limited thereto. The reference electrode 253 may be accommodated in the second body 252, and the ground electrode 254 may be accommodated in the first body 251.

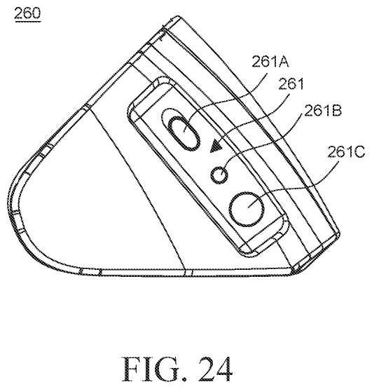

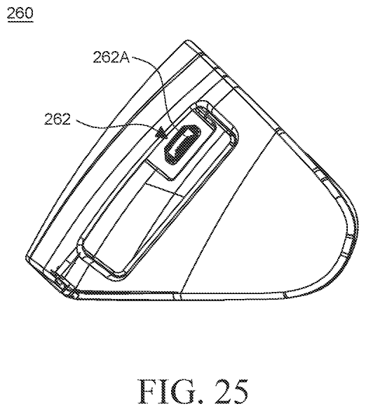

FIG. 24 is a view illustrating a state of a control unit of the biosignal sensing device according to another embodiment of the present invention when viewed from the left side, and FIG. 25 is a view illustrating a state of the control unit of the biosignal sensing device according to another embodiment of the present invention when viewed from the right side.

Referring to FIGS. 24 and 25, the biosignal sensing device 200 may further include the control unit 260.

The control unit 260 may be fixed by being coupled to the third coupling portion 224C of the second bracket 224 coupled to the head-mounted display device HMD′.

The control unit 260 may be electrically connected to the sensor 210 and receive at least one biosignal detected by the sensor 210. The control unit 260 may process and convert the received biosignal and transmit the biosignal to an external device.

The control unit 260 may include a first controller 261 and a second controller 262.

The first controller 261 may be provided at one side of the control unit 260. The first controller 261 may include a power switch 261A configured to manage power for the control unit 260, a reset switch 261B configured to reset the control unit 260, and a first connection terminal 261C configured to accommodate the connection cable of the ear clip 250 so that the ear clip 250 and the control unit 260 are electrically connected.

The second controller 262 is disposed at the other side opposite to one side where the first controller 261 is disposed. The second controller 262 may include a second connection terminal 262A.

The second connection terminal 262A may accommodate a connection cable provided in the external device. Accordingly, the digital signal may be transmitted from the control unit 260 to the external device such as the user terminal, the head-mounted display device, or the server through the second connection terminal 262A.

In addition, the control unit 260 may amplify and process at least one biosignal detected by the sensor 210 and perform A/D conversion on the processed biosignal.

Specifically, the control unit 260 may include: a amplifying part configured to amplify the biosignal detected by the sensor 210; a signal processing part configured to remove noise from the amplified signal; a power supplying part configured to supply power; an A/D conversion part configured to convert the nosie-removed biosignal into a digital signal; a communication part connected to the external device in a wired or wireless manner and configured to transmit the biosignal converted into the digital signal to the external device; and a controller configured to control the above-mentioned components.

Although the embodiments of the present invention have been described in detail with reference to the accompanying drawings, the present invention is not limited thereto and may be embodied in many different forms without departing from the technical concept of the present invention. Therefore, the embodiments of the present invention are provided for illustrative purposes only but not intended to limit the technical concept of the present invention. The scope of the technical concept of the present invention is not limited thereto. Therefore, it should be understood that the above-described embodiments are illustrative in all aspects and do not limit the present invention. The protective scope of the present invention should be construed based on the following claims, and all the technical spirit in the equivalent scope thereto should be construed as falling within the scope of the present invention.

What is claimed is:

1. A biosignal sensing device comprising:
a sensor configured to detect at least one biosignal while being in contact with a user's skin,
wherein the sensor is deformed to a shape corresponding to a contact portion and comes into close contact with the contact portion.

2. The biosignal sensing device of claim 1, wherein the sensor is disposed on the user's forehead portion and measures the at least one biosignal.

3. The biosignal sensing device of claim 1, wherein the sensor comprises:
a plurality of electrodes configured to detect the at least one biosignal while being in contact with the user's skin;
a flexible printed circuit board electrically connected to the plurality of electrodes and configured to be deformed to correspond to a shape of the contact portion; and
a connection terminal electrically connected to the flexible printed circuit board and an external device.

4. The biosignal sensing device of claim 3, wherein the plurality of electrodes comprises:
a reference electrode;
a plurality of active electrodes disposed to be opposite to one another and provided at one side and the other side of the reference electrode based on a horizontal direction with respect to the reference electrode; and
a plurality of ground electrodes disposed to be opposite to one another and provided at one side and the other side of the reference electrode based on the horizontal direction with respect to the reference electrode, and
wherein the plurality of active electrodes is disposed at least two positions based on vertical direction.

5. The biosignal sensing device of claim 4, wherein the plurality of active electrodes comprises:
a first active electrode disposed above the reference electrode based on the vertical direction;
a second active electrode disposed below the reference electrode based on the vertical direction; and
a third active electrode disposed below the second active electrode based on the vertical direction, and
wherein the first active electrode, the second active electrode, and the third active electrode are disposed at different positions based on the horizontal direction.

6. The biosignal sensing device of claim 3, further comprising:
a bracket configured to be coupled to a head-mounted display device; and
a support pad coupled to the bracket and configured to bring the sensor coupled to one side into close contact with the user's skin.

7. The biosignal sensing device of claim 6, wherein the bracket comprises:
a support plate coupled to the support pad and having a curved shape; and
a coupling member extending from the support plate and configured to be coupled to the head-mounted display device.

8. The biosignal sensing device of claim 6, wherein the support pad has a concave-convex structure configured to elastically support the plurality of electrodes.

9. The biosignal sensing device of claim 8, wherein the support pad comprises:
a base coupled to the bracket and configured to maintain a shape corresponding to the bracket; and
a plurality of pressing protrusions provided on the base so that the plurality of electrodes each disposed on one surface of each of the plurality of pressing protrusions is disposed at positions spaced apart from one surface of the base, the plurality of pressing protrusions being configured to elastically support the plurality of electrodes and bring the plurality of electrodes into close contact with the user's skin.

10. The biosignal sensing device of claim 9, wherein the support pad is made of a material having elasticity, and the plurality of pressing protrusions each have a shell structure having a vacant internal space.

11. The biosignal sensing device of claim 6, further comprising:
a control unit electrically connected to the sensor and configured to receive the at least one biosignal detected by the sensor, process and convert the received biosignal, and transmit the biosignal to the external device.
